(19) United States
(12) Patent Application Publication
(10) Pub. No.: US 2022/0211551 A1
(43) Pub. Date: Jul. 7, 2022

(54) HYGIENE MONITORING DEVICE WITH NARROW DATA LOGGING UNIT

(71) Applicant: Essity Hygiene and Health Aktiebolag, Göteborg (SE)

(72) Inventor: Josefin AMBRING, Göteborg (SE)

(73) Assignee: Essity Hygiene and Health Aktiebolag, Göteborg (SE)

(21) Appl. No.: 17/606,896

(22) PCT Filed: May 16, 2019
(86) PCT No.: PCT/EP2019/062670

§ 371 Date: Oct. 27, 2021

(51) Int. Cl. A61F 13/42; G16H 40/63; G01N 27/02

(52) U.S. Cl. CPC A61F 13/42; A61F 2013/424; G01N 27/02; G16H 40/63

(57) ABSTRACT

A hygiene monitoring device includes an elongate flexible sensing panel defining a longitudinal axis and a transversal axis and having a longitudinal length that is longer than its transversal width, and a data logger unit attached to the flexible sensing panel. The data logger unit defines a longitudinal axis and a transversal axis, and has a longitudinal length that is longer than its transversal width, and, when the data logger unit is attached to the sensing panel, the longitudinal axis of the flexible sensing panel is aligned along the longitudinal axis of the data logger unit. The flexible sensing panel includes a flexible substrate which carries a first sensing element, wherein a fastening material is disposed at a first surface of the flexible sensing panel and said data logger unit is disposed on the opposing, second surface of the flexible sensing panel.

### FIELD OF THE INVENTION

The present invention relates to a hygiene monitoring device comprising an elongate flexible sensing panel defining a longitudinal axis and a transversal axis and being longer in the longitudinal direction than it is wide in the transverse direction, and a data logger unit attached to, or removably attachable to, the flexible sensing panel, said data logger unit defining a longitudinal axis and a transversal axis, and having a longitudinal length that is longer than its transversal width.

The present invention also relates to a system comprising a hygiene monitoring device and at least one hygiene absorbent article.

### BACKGROUND

Absorbent articles, such as diapers, absorbent underwear, sanitary products and incontinence shields require periodic replacement, in use, to ensure that the absorbency of the article is not compromised.

In many settings, including domestic settings, institutional settings, healthcare settings and the like, there is a need to monitor the state of an absorbent article provided to a user to ensure that the article contains satisfactory absorbent capacity to fulfil its function.

Conventionally, such monitoring may be performed as self-monitoring by the user, in which the user informs care staff that the capacity of the absorbent article to absorb has diminished, or by periodic checking of the absorbent article by the user or by care staff. However, such processes are labour intensive. Moreover, the information gathered about the personal needs of the user in respect of the needed frequency of replacement of the absorbent article or the needed capacity of the absorbent article is slow to aggregate and is frequently and incompletely collected.

Accordingly, it has been proposed to provide absorbent articles with sensors, which are coupled to data logging electronics. The data logging electronics can determine the absorbent state of the absorbent article, and, for example, can notify a carer when the absorbent state of the absorbent article has reached a predetermined state.

Arrangements have been proposed which use sensing wires embedded in an absorbent core of the absorbent article, such that data logging electronics measure the resistance between the wires to determine the presence of liquid in the absorbent core, and hence the state of the absorbent article.

However, because such sensors are difficult to integrate into the absorbent core of an absorbent article, the sensing capability may be reduced in systems using replaceable logging packages. Moreover, since absorbent articles are often provided in a range of forms and sizes, a logging package which is appropriate for provision to one type or size of absorbent article may perform poorly when used in combination of another type or size of absorbent article.

Arrangements have also been proposed which use removably attachable sensors to the outer surface of the absorbent product. Such arrangement may be beneficial from a sustainability and recycling point of view as the electronics and sensor wires need not be disposed together with the absorbent article but may instead be reused and/or disposed of separately.

However, such externally positioned sensors have shown disadvantages in reducing the wearing comfort of the absorbent articles, as well as being prone to fall off the absorbent article during use, leading to incomplete or misleading acquisition of information.

Accordingly, there is need for means of measuring and monitoring the absorbent status of absorbent articles which overcomes at least some of the drawbacks associated with prior arrangements.

### SUMMARY

According to a first aspect of the present invention, there is provided a hygiene monitoring device comprising an elongate flexible sensing panel and a data logger unit. The elongate flexible sensing panel defines a longitudinal axis and a transversal axis and is longer in the longitudinal direction than it is wide in the transverse direction. The data logger unit is attached to, or is removably attachable to, the flexible sensing panel and defines a longitudinal axis and a transversal axis and has a longitudinal length that is longer than its transversal width. When the data logger unit is attached to the sensing panel, the longitudinal axis of the flexible sensing panel is substantially aligned along the longitudinal axis of the data logger unit. The flexible sensing panel comprises a flexible substrate which carries at least a first sensing element. A fastening material is disposed at a first surface of the flexible sensing panel and the data logger unit is disposed on the opposing, second surface of the flexible sensing panel.

The transverse width of the data logger unit may be at most 160, 150, 140, 130, 120 or 110% of the maximum transverse width of the flexible sensing panel within the longitudinal extent of the flexible sensing panel covered by the data logger unit.

The transverse width of the data logger unit may be at least 80, 90 or 100% of the transverse width of the flexible sensing panel within the longitudinal extent of the flexible sensing panel covered by the data logger unit.

The ratio of the transverse width of the flexible sensing panel, within the longitudinal extent of the flexible sensing panel covered by the data logger unit, to the longitudinal length of the sensing panel may be from 0.025 to 0.1.

The transverse width of the flexible sensing panel, within the longitudinal extent of the flexible sensing panel covered by the data logger unit, may be from 20 or 22 to 30 or 28 mm, such as about 25 mm.

The longitudinal length of the flexible sensing panel may be from 200, 300, 400 or 500, to 800, 700, 600, 500 or 400 mm, such as from 200 to 400 mm or from 500 to 700 mm.

Over at least 60, 70, 80, 90 or 95% of its longitudinal extension, the flexible sensing panel may have substantially straight and parallel longitudinally extending side edges.

The ratio of the transverse width to the longitudinal length of the data logger unit may be from 0.3 to 0.8.

The transverse width of the data logger unit may be from 20, 25 or 30 to 45 or 40 mm, such as about 35 mm.

The longitudinal length of the data logger unit may be from 30, 35, 40, 45 or 50 to 70, 65, 60 or 55 mm, such as about 50 to 55 mm.

The data logger unit may be removably attachable to the sensing panel, and the thickness of the data logger unit, in the direction orthogonal to the longitudinal axis and the transversal axis of the data logger unit, may be from 2, 5, 8 or 10 to 20 or 15 mm, such as about 13 mm.

The hygiene monitoring device may be adapted for being removably attached to a garment facing side of an absorbent hygiene product by means of the fastening material. The absorbent hygiene product defines a body facing side, intended to face the body of the wearer during use, and an opposing garment facing side, intended to face away from the wearer during use.

The absorbent hygiene product may be a diaper such as a pant-type diaper, an open-type diaper, a belt-type diaper, or an incontinence pad or a sanitary napkin.

The hygiene monitoring device may be adapted to detect the presence of body fluids absorbed by the absorbent hygiene product.

When the data logger unit is attached to the sensing panel, it may be electrically connected to the at least one sensing element.

A sensing element according to the present disclosure may comprise a first sensing plate and a second sensing plate plates and the hygiene monitoring device may be configured to measure the impedance between said first and second sensing plates.

The fastening material may comprise a hook material for removably securing the sensing panel to a fibrous nonwoven material at the garment facing side of the absorbent hygiene product (20).

The fastening material may cover at least 90%, such as from 90 to 95 to 100 or 99% of the first surface of the sensing panel.

The fastening material may form a continuous area on the first surface of the sensing panel.

When attached to the flexible sensor panel, the longitudinal center of the data logger unit may be positioned closer to a longitudinally outermost edge of the sensor panel than to the longitudinal center of the flexible sensor panel.

The flexible sensing panel max extend longitudinally beyond both longitudinal outermost edges of the data logger unit.

The distance from the longitudinally outermost edge of the data logger unit to the closest one of the longitudinally outermost edges of the flexible sensing panel may be at least 10 mm, such as from 10, 20 or 30 to 60, 50 or 40 mm.

When attached to the flexible sensor panel the transversal center of the data logger unit may be substantially super positioned on the transversal center of the flexible sensing panel.

In a device according to the present disclosure, a panel-side logger attachment means may be arranged on the second surface on the flexible sensing panel and a logger-side logger attachment means may be arranged on the data logger unit. The panel-side logger attachment means and the logger-side logger attachment means are adapted to removably attach the data logger unit to the flexible sensing panel and to electrically connect the data logger unit with the at least one sensing element.

When attached to the flexible sensor panel, the data logger unit may abut the second surface of the flexible sensing panel.

The present disclosure also relates to a system comprising a hygiene monitoring device as disclosed herein, and at least one absorbent hygiene article.

In such system, the hygiene monitoring device may be is removably secured or securable on a garment facing side of the absorbent hygiene article.

### BRIEF DESCRIPTION OF THE DRAWINGS

For a better understanding of the invention, and to show how the same may be carried into effect, reference will now be made, by way of example, to the accompanying drawings, in which:

FIG. 1 is a schematic top view of a flexible sensing panel according to one embodiment of the invention;

FIG. 2 is a schematic top view of a data logger unit according to one embodiment of the invention;

FIG. 3 is a schematic top view of a hygiene monitoring device with a data logger arranged on a flexible sensing panel according to one embodiment of the invention;

FIG. 4 is a schematic side-view of a hygiene monitoring device with a data logger arranged on a flexible sensing panel according to one embodiment of the invention.

FIG. 5 shows a schematic drawing of a hygiene monitoring device secured on the garment facing side of an absorbent hygiene product according to one embodiment of the invention.

### DETAILED DESCRIPTION

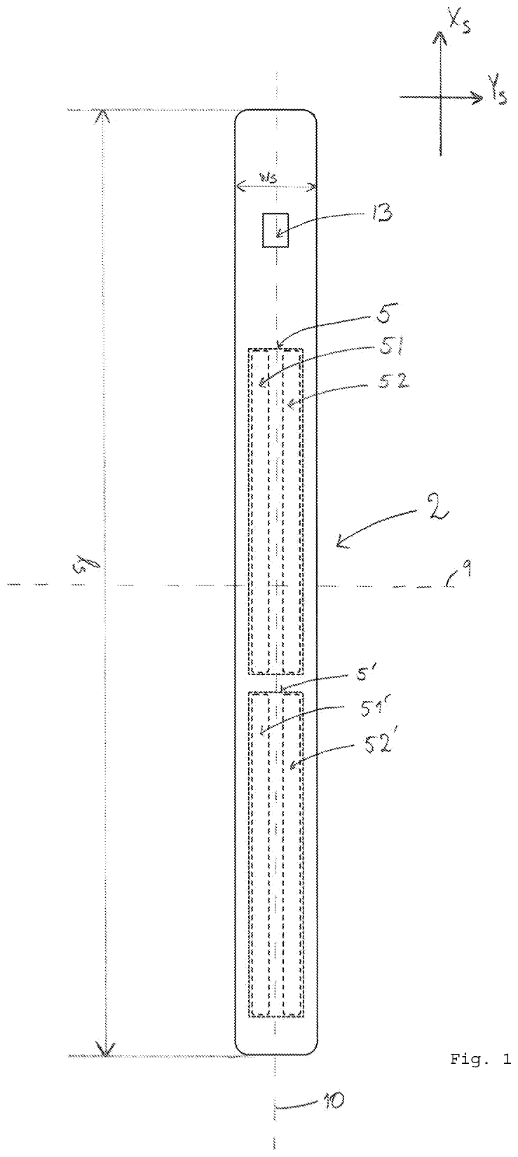

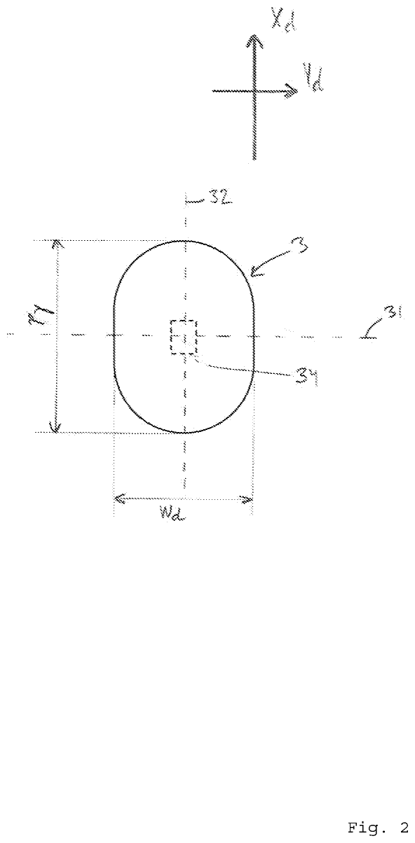

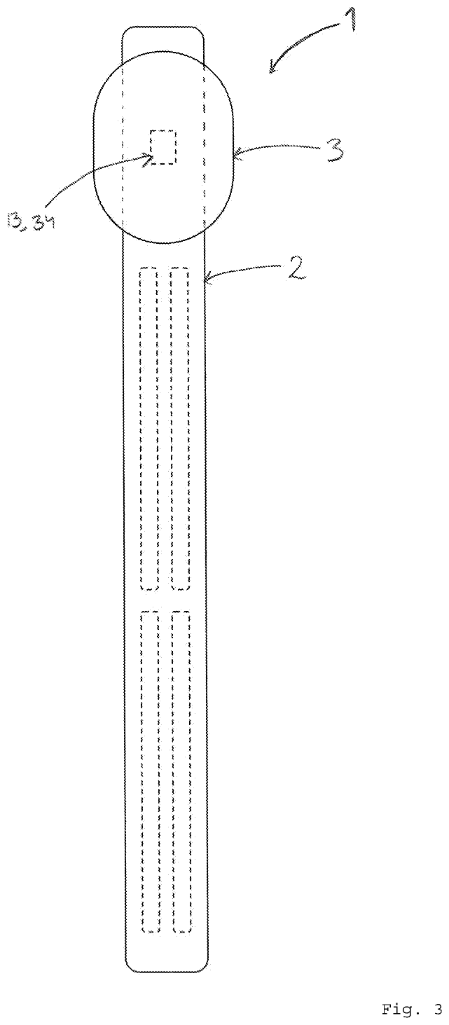

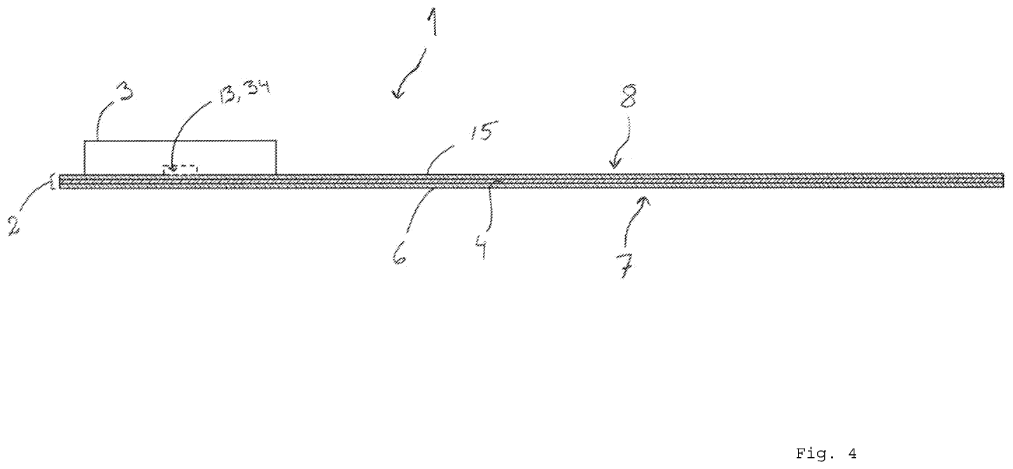

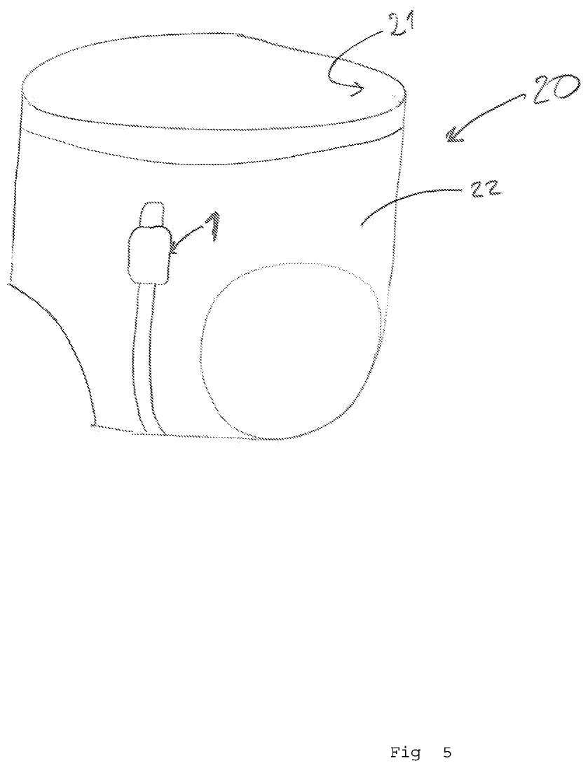

A hygiene monitoring device 1 of the present disclosure comprises a flexible sensing panel 2 and a data logger unit 3 attached to, or removably attachable to the flexible sensing panel 2. FIGS. 1 and 2 shows the flexible sensing panel 2 and the data logger unit 3 in isolation from each other, while FIGS. 3 and 4 show the hygiene monitoring device 1 with the flexible sensing panel 2 and the data logger unit attached to each other, and FIG. 5 shows a hygiene monitoring device secured on the garment facing side 22 of an absorbent hygiene product 20.

As shown in FIG. 1, flexible sensing panel 2 has a length along the longitudinal axis Xs longer that the width along the transverse axis Ys, giving it a generally elongate shape. While the sensing panel in FIG. 1 has a generally rectangular shape, other shapes of the flexible sensing panel are also possible within the scope of the present disclosure.

The length lₛ of the flexible sensing panel 2 will depend on the size of the absorbent product on which it is to be secured, but may vary from about 200 to about 700 mm.

The transversal width wₛ is measured in proximity to the sensing-panel side logger attachment means 13 to represent the maximum width of the flexible sensing panel 2 in the longitudinal extent thereof that will be covered by a data logger unit 3, when the data logger unit 3 is attached to the flexible sensing panel, and may be from 20 or 22 to 30 or 28 mm, such as about 25 mm. The ratio of the transverse width wₛ to the longitudinal length lₛ of the sensing panel may be from 0.025 to 0.1

Towards one of the longitudinal edges of the flexible sensing panel a panel-side logger attachment means 13 is disposed for connecting the flexible sensing panel 2 to a data logger unit 3, which is to be described below.

The flexible sensing panel 2 is generally made from one or more flexible, easily bendable materials, but may comprise rigid components, such as the panel-side logger attachment means 13.

As is shown in FIG. 4, the flexible sensing panel 2 comprises a flexible substrate 4 such as flexible printed circuit board (flex-PCB). The flexible substrate 4 carries a first sensing element 5 and a second sensing element 5′ that are adapted to be used for detecting the presence of body liquids absorbed in a hygiene absorbent product to which the hygiene monitoring device 1 is secured. The sensing elements 5, 5′ may be printed on the flexible substrate or otherwise arranged thereon. The sensing elements 5, 5′ are electrically connected by conductive leads (not shown) to the panel-side logger attachment means 13.

As can be viewed in FIG. 4, on the side of the flexible substrate 4 opposing that of the panel-side logger attachment means 13, a fastener material 6 is disposed. The fastener material 6 is adapted to removably secure the flexible sensing panel 2 to an absorbent hygiene product. This may for example be obtained by means of hooks for fastening to a loop material, such as a nonwoven, on the absorbent hygiene product, or by means of an adhesive for fastening to plastic film surface on the absorbent hygiene product. Hook materials suitable for use include hook tapes obtainable from commercial sources, such as Velcro and 3M. The fastening material 6 advantageously covers essentially the full area, or at least 95, or at least 90% of that side of the flexible substrate.

On the side of the flexible substrate 4 opposite to the fastening material, i.e. on the side of the panel-side logger attachment means 13, a protective cover layer 15, such as a tape or a coating layer, may optionally be arranged to protect the flexible substrate 4 and/or to provide a suitable surface structure.

FIG. 2 shows an exemplary configuration of a data logger unit 3 being one component of a hygiene monitoring device 1 shown I FIGS. 3 and 4. The data monitoring unit 3 is adapted to be removably attachable to the flexible sensing panel 2.

The data logger unit 3 has a longitudinal length l_{d} that is longer than its transverse width w_{d}, to obtain an elongate shape, for example with an oblong-like shape

The data logger unit 3 comprises a logger-side logger attachment means 34 that is adapted to interact with the panel-side logger attachment means 13.

The logger-side logger attachment means 34 and the panel-side logger attachment means are arranged such that when the data logger unit 3 is attached to the flexible sensing panel, the longitudinal axis Xd of the logger unit 3 is arranged substantially along the longitudinal axis Xs of the flexible sensing panel 2.

The width w_{d} of the data logger unit 3 may be up to 160% of the width wₛ of the flexible sensing panel. Therefore, by allowing the data logger unit 3 to be relatively narrow, it will not significantly reduce the wearing comfort of an absorbent product equipped with a hygiene monitoring device 1 of the present disclosure, as the data logger unit 3 will not add a significant amount of comfort reduction.

The width w_{d} of the data logger unit 3 may also be at least 80% of the of the width wₛ of the flexible sensing panel. Therefore, by allowing the data logger unit 3 to be relatively close in width to the underlying flexible sensing strip, it will be able to be clearly recognizable and easy to grab when it is to be removed from the flexible sensing strip 2.

The ratio of the transverse width w_{d} to the longitudinal length l_{d} of the data logger unit 3 may be from 0.3 to 0.8.

The transverse width w_{d} of the data logger unit may be from 20, 25 or 30 to 45 or 40 mm, such as about 35 mm.

The longitudinal length l_{d} of the data logger unit may be from 30, 35, 40, 45 or 50 to 70, 65, 60 or 55 mm, such as about 50 to 55 mm.

The thickness of the data logger unit 3, in the direction orthogonal to the longitudinal axis Xd and the transversal axis Yd of the data logger unit may preferably be less than 20 mm, such as from 2, 5 or 8 to 20, 15 or 10 mm. A low thickness reduces the discomfort for the user during use of the absorbent product 20 when a hygiene monitoring device 1 is secured thereto.

The longitudinal center 31 of the data logger unit 3, represented by a transversal line dividing the data logger unit 3 in two equally long portions, may be positioned closer to a longitudinally outermost edge of the flexible sensing panel 2 than to the longitudinal center 9, represented by a transversal line dividing the data logger unit in two equally long portions of the flexible sensor panel. This positions the data logger unit 3 fairly close to one of the longitudinal edges of the flexible sensing panel 2 and allows for a low impact on the wearer comfort.

The distance from the longitudinally outermost edge of the data logger unit 3 to the closest one of the longitudinally outermost edges of the flexible sensing panel 2 may be at least 10 mm.

This allows for the formation grip tab portion of the flexible sensing panel allowing easy securing and removal of the hygiene monitoring device to an absorbent article.

The transversal center 32 of the data logger unit 3 may, as shown in FIG. 3, be substantially super positioned on the transversal center 10 of the flexible sensing panel 2a.

When the data logger unit 3 is attached to the flexible sensing panel 2 by means of the logger-side logger attachment means 34 and the panel-side logger attachment means 13, the data logger unit 3 may abut the second surface 8 of the flexible sensing panel 2.

When, as is shown in FIGS. 3 and 4, the data logger unit 3 is attached to the flexible sensing panel 2 by means of the logger-side logger attachment means 34 and the panel-side logger attachment means 13 a physical attachment is obtained while also electrically connecting the electronic circuitry housed in the data logger unit with the sensing elements 5, 5′ on the flexible sensing panel 2.

The logger-side logger attachment means 34 and the panel-side logger attachment means 13 may be any connector combination as known to those in the art, such as, mezzanine connectors, plug-and-socket connectors, registered jack or modular connectors, pogo-pin connectors, tip-ring-sleeve connectors, D-subminiature connectors, DIN connectors, or other terminal or connector types as known in the art. Where such connectors are gendered, either of the male part or the female part may be provided to data logger unit 3 and the other of the male part or the female part may be provided to sensing panel 2. Such terminal arrangements may also have a function to retain data logger unit 3 and sensing panel 2 in physical engagement as well as electrical connection, especially when locking or otherwise secured variants of such connectors are provided. However, the electrical connection and physical attachment may be obtained by two or more attachment means interacting to form the attachment means 13, 34.

When the electronic circuitry housed in the data logger unit 3 is electrically connected to the sensing elements 5, the hygiene monitoring device 1 is operable and capable of detecting the presence of, and optionally the quantity of, body liquids absorbed in an absorbent hygiene product which the hygiene monitoring device 1 is secured.

The electronic circuitry may comprise a power source, such as a cell battery or a rechargeable battery, sensor-driving electronics for driving the sensing elements 5, 5′ and a transmitter for transmitting data such as sensor values and optionally other information to an external device, such as a computer or a hand-held device. The transmitter preferably transmits data by means of a wireless protocol, such as Zigbee, WiFi or Bluetooth. The electronic circuitry may also comprise a receiver for receiving data from an external device.

In the exemplary configuration shown in FIG. 1, each sensing element comprises a first sensing plate 51, 51′ and a second sensing plate 52, 52′. The detection of body liquids involves measuring the impedance between the first sensing plate 51, 51′ and the second sensing plate 52, 52′ and detecting a change in the impedance value over time. However, the present disclosure is not limited to this specific measuring technique, and may rely on other types of sensors, such as sensors measuring relative humidity, VOC (volatile organic compounds) and/or other gasses, temperature, conductivity etc. However, in embodiments, such as in the case of the impedance measurements, the detection of body liquids may advantageously be performed without requiring direct contact between the sensing panel and the body liquids, especially without requiring galvanic contact between the sensing elements 5, 5′ and the body liquid to be detected.

FIG. 5 shows an exemplary configuration of a hygiene monitoring device 1 secured on the outside of an absorbent hygiene product 20, defining an inner, body facing side 21 and an outer garment facing side 21. The absorbent hygiene product 20 typically comprises an absorbent core to absorb body fluids such as urine, faces and/or menstrual fluid discharged from the wearer while wearing the absorbent hygiene product 20. The absorbent core is typically arranged between a liquid permeable top-sheet and a substantially liquid impermeable back-sheet as is conventional in the art.

In FIG. 5, the hygiene absorbent product 20 is shown as a pant-type diaper, however any type of hygiene absorbent product configured to be worn in the crotch area of a wearer may be used with a hygiene monitoring device 1 of the present disclosure, such as open-type diapers, belt-type diapers, pant-type diapers, incontinence shields (pads) or sanitary napkins.

The garment facing side 22 of the hygiene absorbent product 20 may have a surface of fibrous non-woven material, to which a hook-type fastening material 6 of the sensing panel 2 may readily adhere in order to releasably secure the hygiene monitoring device 1 on the hygiene absorbent article 20. The garment facing side 22 of the hygiene absorbent product 20 may alternatively have a plastic film surface, and the fastening material 6 may be an adhesive, such as a pressure sensitive adhesive, in order to releasably secure the hygiene monitoring device 1 on the hygiene absorbent article 20.

In FIG. 5, the hygiene monitoring device 1 is secured on the hygiene absorbent product 20 with the data logger unit 2 towards a front waist region of the hygiene absorbent product 20, and the flexible sensing panel 2 extending towards the rear waist region of the hygiene absorbent product 20. Alternative positions of the hygiene monitoring device are also possible, such as, but not limited to, with the data logger unit arranged towards a rear waist region of the hygiene absorbent product and the flexible sensing panel 2 extending towards the front waist region of the hygiene absorbent product.

By arranging the data logger unit 3 with its longitudinal axis Xd along the longitudinal axis Xs of the flexible sensing panel 2 the area of the data logger unit 3 is distributed over a larger area of the flexible sensing panel 3. When the hygiene monitoring device is secured on the garment facing side 22 of the hygiene absorbent product 20, the device of the present disclosure therefore reduces the risk of the hygiene monitoring device to fall of the absorbent hygiene product, as the weight of the data logger unit is distributed over a larger area of the fastening material 6.

In another aspect, the present disclosure relates to a system comprising a hygiene monitoring device 1 as disclosed herein, and at least one absorbent hygiene article 20.

In such system, the hygiene monitoring device 1 may be removably secured on a garment facing side 22 of the absorbent hygiene article 20, or it may be provided as a separate entity from the absorbent hygiene article 20. For example, a hygiene monitoring device 1 may be provided in a package containing one or more absorbent hygiene articles 20. In another alternative, a data logger unit 3, one or more sensing panels 2 and one or more absorbent articles 20 may be provided as a system.

1. A hygiene monitoring device, comprising an elongate flexible sensing panel defining a longitudinal axis and a transversal axis and being longer in the longitudinal direction than it is wide in the transverse direction, and a data logger unit attached to, or removably attachable to, the flexible sensing panel, said data logger unit defining a longitudinal axis and a transversal axis, and having a longitudinal length that is longer than its transversal width, and, when the data logger unit is attached to the sensing panel, the longitudinal axis of the flexible sensing panel is aligned along the longitudinal axis of the data logger unit, wherein the flexible sensing panel comprises a flexible substrate which carries at least a first sensing element, wherein a fastening material is disposed at a first surface of the flexible sensing panel and wherein said data logger unit is disposed on the opposing, second surface of the flexible sensing panel.

2. The device according to claim 1, wherein the transverse width of the data logger unit is no more than 160% of the maximum transverse width of the flexible sensing panel within the longitudinal extent of the flexible sensing panel covered by the data logger unit.

3. The device according to claim 1, wherein the transverse width of the data logger unit is at least 80% of the transverse width of the flexible sensing panel within the longitudinal extent of the flexible sensing panel covered by the data logger unit.

4. The device according to claim 1, wherein the ratio of the transverse width, within the longitudinal extent of the flexible sensing panel covered by the data logger unit, to the longitudinal length of the sensing panel is from 0.025 to 0.1.

5. The device according to claim 1, wherein the transverse width of the flexible sensing panel, within the longitudinal extent of the flexible sensing panel covered by the data logger unit, is from 20 to 30 mm.

6. The device according to claim 1, wherein the longitudinal length of the flexible sensing panel is from 200 to 700 mm.

7. The device according to claim 1, wherein the flexible sensing panel, over at least 60% of its longitudinal extension, has substantially straight and parallel longitudinally extending side edges.

8. The device according to claim 1, wherein the ratio transverse width/longitudinal length of the data logger unit is from 0.3 to 0.8.

9. The device according to claim 1, wherein the transverse width of the data logger unit is from 20 to 45 mm.

10. The device according to claim 1, wherein the longitudinal length of the data logger unit is from 30 to 70 mm.

11. The device according to any one of the preceding claims claim 1, wherein the data logger unit is removably attachable to the flexible sensing panel, and the thickness of the data logger unit, in the direction orthogonal to the longitudinal axis and the transversal axis of the data logger unit, is from 2 to 20 mm.

12. The device according to claim 1, is adapted for being removably attached to a garment facing side of an absorbent hygiene product by means of said fastening material, said absorbent hygiene product defining a body facing side, configured to face the body of the wearer during use, and an opposing garment facing side, configured to face away from the wearer during use.

13. The device according to claim 1, wherein said absorbent hygiene product is a diaper.

14. The device according to claim 1, wherein the device is configured to detect the presence of body fluids absorbed by the absorbent hygiene product.

15. The device according to claim 1, wherein the data logger unit, when attached to the sensing panel, is electrically connected to the at least one sensing element.

16. The device according to claim 1, wherein said sensing element comprises a first sensing plate and a second sensing plate, and the device is configured to measure an impedance between said first and second sensing plates.

17. The device according to claim 1, wherein the fastening material comprises a hook material for removably securing the sensing panel to a fibrous nonwoven material at the garment facing side of the absorbent hygiene product.

18. The device according to claim 1, wherein the fastening material covers at least 90% of the first surface of the sensing panel.

19. The device according to claim 1, wherein the fastening material forms a continuous area on the first surface of the sensing panel.

20. The device according to claim 1, wherein, when attached to the flexible sensor panel, the longitudinal center of the data logger unit is positioned closer to a longitudinally outermost edge of the sensor panel than to a longitudinal center of the flexible sensor panel.

21. The device according to claim 1, wherein the flexible sensing panel extends longitudinally beyond both longitudinal edges of the data logger unit.

22. The device according to claim 1, wherein a distance from a longitudinally outermost edge of the data logger unit to a closest one of the longitudinally outermost edges of the flexible sensing panel is at least 10 mm.

23. The device according to claim 1, wherein, when attached to the flexible sensor panel, the transversal midpoint of the data logger unit is substantially super positioned on the transversal midpoint of the flexible sensing panel.

24. The device according to claim 1, wherein a panel-side logger attachment is arranged on the second surface on the flexible sensing panel, where a logger-side logger attachment is arranged on the data logger unit, and wherein the panel-side logger attachment and the logger-side logger attachment are adapted to removably attach the data logger unit to the flexible sensing panel and to electrically connect the data logger unit with the at least one sensing element.

25. The device according to claim 1, wherein the data logger unit abuts the second surface of the flexible sensing panel.

26. A system comprising a hygiene monitoring device as defined in claim 1 and at least one absorbent hygiene article.

27. The system according to claim 26, wherein said hygiene monitoring device is removably secured on a garment facing side of the absorbent hygiene article.
